(19) United States
(12) Patent Application Publication
(10) Pub. No.: US 2016/0235329 A1
(43) Pub. Date: Aug. 18, 2016

(54) NERVE BUNDLE CUFF INCLUDING ELECTRODES AND TRANSDUCERS

(71) Applicant: The Charles Stark Draper Laboratory, Inc., Cambridge, MA (US)

(72) Inventors: Jonathan J. Bernstein, Medfield, MA (US); Anilkumar Harapanahalli Achyuta, Cambridge, MA (US)

(21) Appl. No.: 14/983,998

(22) Filed: Dec. 30, 2015

Related U.S. Application Data
(60) Provisional application No. 62/115,976, filed on Feb. 13, 2015.

(51) Int. Cl. A61B 5/05; A61B 8/00; A61B 5/00; A61B 8/08

(52) U.S. Cl. CPC A61B 5/05; A61B 8/48; A61B 8/4483; A61B 5/6831

(57) ABSTRACT

The present disclosure describes systems and methods for recording and stimulating neural and other tissue. The disclosure describes a tissue interface that can be configured as a cuff or a ribbon and includes a plurality of electrodes and ultrasound transducers. The tissue interface is configured to electrically and ultrasonically stimulate tissue, such a muscle tissue and neural tissue. The tissue interface is also configured to monitor the target tissue by recording electrical activity of the target tissue with one or more of the electrodes and image the target tissue with one or more of the ultrasound transducers.

### CROSS-REFERENCE TO RELATED APPLICATIONS

This application claims priority to and benefit of U.S. Provisional Patent Application No. 62/115,976 filed on Feb. 13, 2015 and titled “NERVE BUNDLE CUFF INCLUDING ELECTRODES AND TRANSDUCERS,” which is herein incorporated by reference in its entirety.

### BACKGROUND OF THE DISCLOSURE

Electrodes can be implanted into target tissue to record from and stimulate the target tissue. For example, depth electrodes can be implanted into the brain for the treatment of Parkinson's disease and epilepsy. The patient's body can encapsulate the electrodes over time with fibrous tissue, reducing their efficacy.

### SUMMARY OF THE DISCLOSURE

According to one aspect of the disclosure, a tissue interface device includes a biocompatible polymer backing. The device also includes an electrode array that is coupled to the biocompatible polymer backing. The electrode array includes one or more electrode shafts that project substantially perpendicular to a plane of the biocompatible polymer backing. Each of the one or more electrode shafts include an electrode site. The device also includes one or more ultrasound transducers coupled to the biocompatible polymer backing.

In some implementations, each of the one or more electrode shafts include a plurality of electrode sites distributed along a length of the respective one or more electrode shafts. The one or more ultrasound transducers can include at least one of a bulk ferroelectric ceramic, a crystal ferroelectric, a ferroelectric polymer, a capacitive micromachined ultrasonic transducer (cMUT), a piezoelectric micromachined transducer (pMUT), and an electrostrictive polymer.

In some implementations, the device includes a controller that is configured to energize the one or more ultrasound transducers at a first frequency for stimulating a target and at a second frequency for imaging the target. In some implementations, the controller is also configured to record an electrical signal detected at the electrode site.

In some implementations, the device includes a rechargeable battery. The device can include an induction coil to wirelessly receive energy from an external source and provide the received energy to the rechargeable battery.

In some implementations, the electrode array includes between about 2 and about 64 electrode shafts. In some implementations, the biocompatible polymer backing is a flexible cuff configured to wrap around a nerve.

According to another aspect of the disclosure, a method includes coupling a tissue interface device to a target tissue. The tissue interface device includes a biocompatible polymer backing and one or more electrodes that are coupled to the biocompatible polymer backing. The device also includes one or more ultrasound transducers that are coupled to the biocompatible polymer backing. The method also includes delivering a stimulation signal to the target tissue via the one or more electrodes and imaging the target tissue via the one or more ultrasound transducers.

In some implementations, the method also includes ultrasonically stimulating the target tissue via the one or more ultrasound transducers. The method can include imaging the target tissue via the one or more ultrasound transducers with a first frequency of ultrasonic energy and ultrasonically stimulating the target tissue via the one or more ultrasound transducers with a second frequency of ultrasonic energy different than the first frequency of ultrasonic energy.

In some implementations, imaging the target tissue includes detecting a scar formation on the target tissue. In some implementations, the tissue interface device is configured as a cuff and coupling the tissue interface device to the target tissue further includes wrapping the tissue interface device around the target tissue.

In some implementations, the one or more electrodes each include an electrode shaft projecting substantially perpendicular to a plane of the biocompatible polymer backing and each electrode shaft includes an electrode site. In some implementations, each electrode shaft includes a plurality of electrode sites distributed along a length of the electrode shaft.

In some implementations, the tissue interface device includes between about 2 and about 64 electrode shafts. In some implementations, the target tissue is neural tissue. In some implementations, the one or more ultrasound transducers include at least one of a bulk ferroelectric ceramic, a crystal ferroelectric, a ferroelectric polymer, a capacitive micromachined ultrasonic transducer (cMUT), a piezoelectric micromachined transducer (pMUT), and an electrostrictive polymer. The method can also include wirelessly charging a battery of the tissue interface device.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates an example system for stimulating and monitoring a target tissue with a tissue interface.

FIG. 2 illustrates the example tissue interface illustrated in the system of FIG. 1.

FIGS. 3A and 3B illustrate an example cuff in a flat and rolled configuration, respectively.

FIGS. 4A and 4B illustrate another example tissue interface in a ribbon configuration with integral ultrasound transducers and electrodes.

FIG. 5 illustrates a flow chart of an example method for stimulating target tissue with a tissue interface.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes systems and methods for recording and stimulating neural and other tissue. The disclosure describes a tissue interface that can be configured as a cuff or a ribbon and includes a plurality of electrodes and ultrasound transducers. The tissue interface is configured to electrically and ultrasonically stimulate tissue, such a muscle tissue and neural tissue. The tissue interface is also configured to monitor the target tissue by recording electrical activity of the target tissue with one or more of the electrodes and image the target tissue with one or more of the ultrasound transducers.

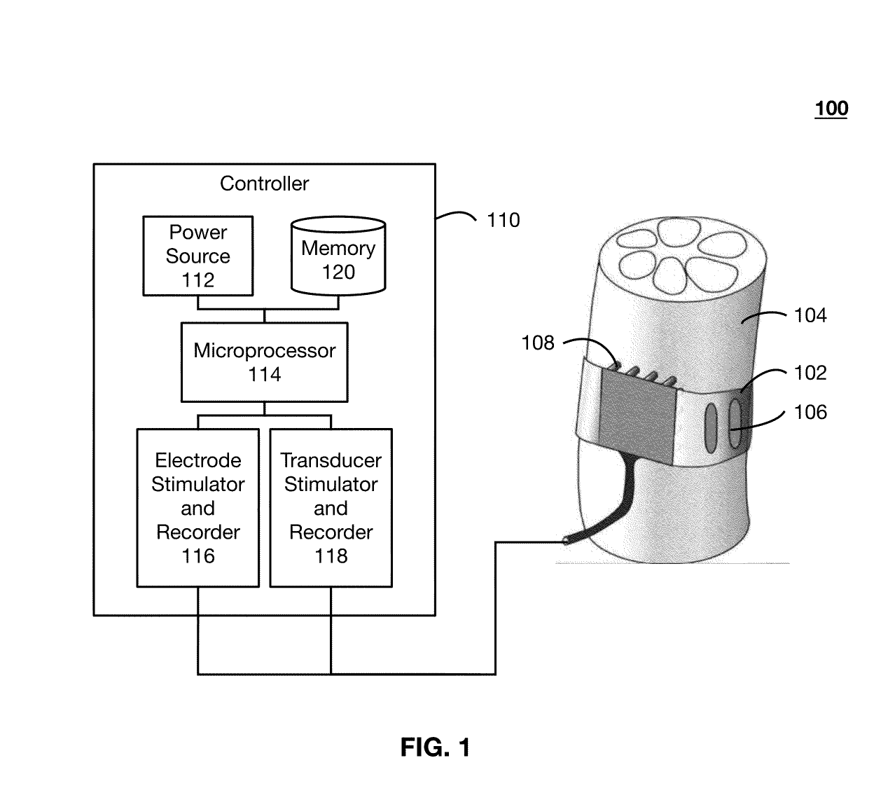

FIG. 1 illustrates an example system 100 for stimulating and monitoring tissue. The system 100 includes tissue interface 102 in a cuff configuration (also referred to as a cuff 102) that is wrapped around a nerve 104, which is also referred to as the target tissue 104. The cuff 102 includes a plurality of ultrasound transducers 106 and a plurality of electrodes 108. The nerve cuff 102 is coupled to and controlled by a controller 110. The controller 110 is powered by a power source 112 and includes a microprocessor 114 that controls an electrode stimulator and recorder (ESR) 116 and a transducer stimulator and recorder (TSR) 118. The data recorded by the ESR 116 and the TSR 118 can be stored in memory 120 for later transfer and analysis.

The controller 110 of the system 100 controls the stimulation and monitoring of the tissue 104. In some implementations, the controller 110 is a hermetically sealed device that is configured for chronic implantation near the target tissue 104. In other implementations, the controller 110 is a handheld device or computer that resides outside of the patient and communicates wirelessly or via a wired connection to the cuff 102. The controller 110 includes one or more microprocessors 114 that control the function of the ESR 116 and the TSR 118. The microprocessor 114 can be any type of single or multi-core processor or special purpose logic circuitry such as an FPGA (field programmable gate array) or an ASIC (application specific integrated circuit). In some implementations, the controller 110 outputs data to other devices for analysis. The controller 110 can communicate with the other devices wirelessly or through a wired connection.

The ESR 116 of the controller 110 generates electrical stimuli that are used to stimulate the tissue 104 at each or a subset of the electrodes 108. For example, the ESR 116 can generate electrical pulses or waves, and can control the frequency, pulse width, signal shape (e.g., square and sinusoidal shaped), amplitude or additional properties of the stimulation, such as selecting which electrodes to stimulate, which to read out, and which ultrasound transducers to actuate. The ESR 116 can generate a stimulation signal with a frequency between about 10 Hz and about 25 kHz, between about 10 Hz and about 10 kHz, between about 100 Hz and about 1 kHz, or between about 100 Hz and about 500 Hz. The ESR 116 also includes one or more analog to digital converters (ADC) that converts the measured electrical activity from the tissue 104 into a digital signal that can be stored in memory 120. The ADCs of the ESR 116 can sample the signal measured by each of the plurality of electrodes 108 at a frequency between about 10 Hz and about 10 kHz, between about 10 Hz and about 5 kHz, between about 10 Hz and about 1 kHz, between about 50 Hz and about 500 Hz, or between about 50 Hz and about 250 Hz. The TSR 118 of the controller 110 generates electrical stimulus that is used to stimulate the transducers 106 and generate ultrasonic energy.

The TSR 118 is configured to generate pulses that cause the transducers 106 to resonate and generate ultrasonic energy between about 0.5 MHz and about 20 MHz, between about 5 MHz and about 15 MHz, or between about 10 MHz and about 15 MHz. In some implementations, the TSR 118 is configured to generates pulses that cause the transducers 106 to resonate and generate ultrasonic energy at a first frequency for stimulating a target and at a second frequency for imaging the target tissue. For example, the stimulation frequency may be a relatively high frequency (e.g., above 10 MHz) when compared to the second frequency used for imaging the target tissue. In some implementations, the frequency for imaging the target tissue is selected such that the ultrasound wave has a wavelength similar to or less than the resolution to needed to image the target tissue. For example, a 5 MHz ultrasound signal has a wavelength (λ) in water of about 0.3 mm using the equation λ=(speed of signal)/(frequency of signal). In some implementations, the ultrasound transducers 106 are used to image the changes that within the target tissue in response to the ultrasonic and/or electrical stimulation to the target tissue. In some implementations, the ultrasound transducers 106 are used to detect the presence of scar tissue that may accumulate around the tissue interface 102 over time. For example, the increase in the acoustic mismatch between the ultrasound transducer 106 and the tissue target can result in a larger echo return, which can signal the development of scar tissue of the tissue target.

The controller 110 of the system 100 also includes a power source 112. When the controller 110 is implanted into a patient, the power source 112 is a battery. In implementations where the controller 110 is external the to the patient, the power source 112 can be a battery or the controller 110 may be plugged into an AC power source (e.g., a wall outlet). In some implementations, the battery of the power source 112 is rechargeable. For example, the controller 110 can include a plurality of induction coils that enable the battery to be wirelessly recharged after the controller 110 is implanted into the patient.

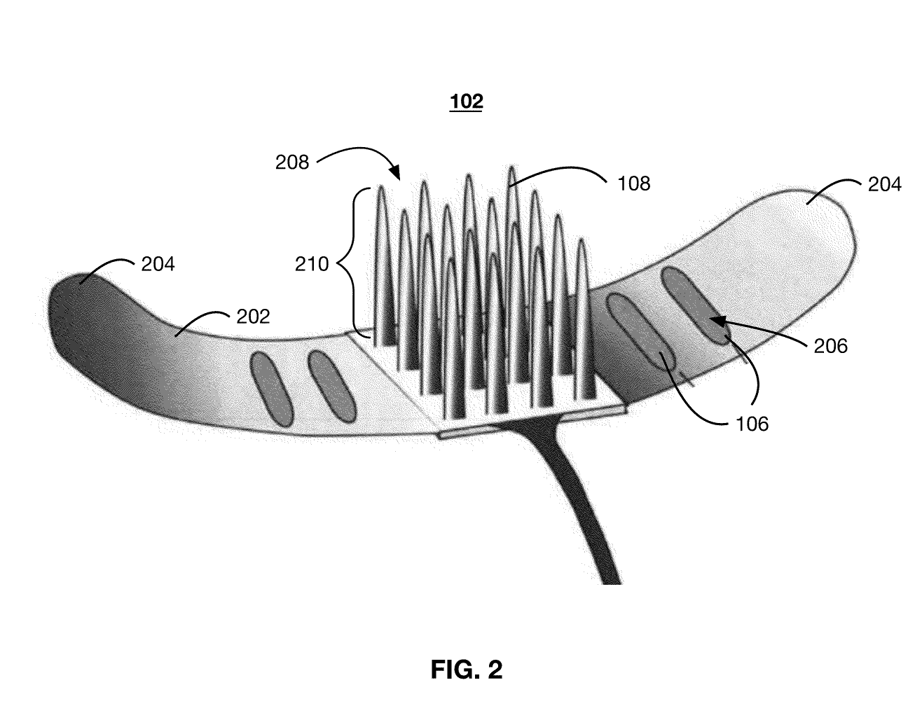

Still referring to FIG. 1, the system 100 also includes a cuff 102. The cuff 102 is described further in relation to FIGS. 2-4, but as an overview the cuff 102 is formed from one or more layers of a biocompatible polymer, such as silicone, poly(dimethyl siloxane) (PDMS), polyurethane, polytetraflouroethylene (PTFE), polyethylene, polypropylene, PMMA, Ethylene-co-vinylacetate, poly(ethylene terphthalate), polysulphone, polyethyleneoxide, or any combination thereof. A surface of the cuff 102 includes a plurality of transducers 106 and a plurality of electrodes 108. The electrodes 108 of the cuff 102 are configured to record and electrically stimulate the tissue 104. The cuff 102 also includes a plurality of transducers 106 to ultrasonically stimulate and image the tissue 104. In some implementations, one or more of the components and functions of the controller 110 can be incorporated into the cuff 102. For example, the cuff 102 can include integrated circuits and other electronics that perform signal filtering, amplification, analog to digital conversion, and channel selection. In some implementations, the cuff 102 is self-contained and also includes the power source 112 (e.g., a rechargeable battery) and an antenna. The implanted cuff 102 can communicate wirelessly with the controller 110 or other device located external to the patient. The implanted, self-contained cuff 102 can also include an induction coil that enables a rechargeable battery to be recharged through magnetic coupling.

FIG. 2 illustrates the example cuff 102 of FIG. 1 prior to implantation. The cuff 102 includes a plurality of transducers 106 embedded within a polymer backing 202. The top layer of the polymer backing 202 can be machined to expose the face 206 of each of the transducers 106. In other implementations, the polymer backing 202 is not machined to expose the face 206 of each of the transducers. For example, the polymer backing 202 can be configured to have approximately the same acoustic impedance as tissue so that the polymer backing 202 does not substantially affect the ultrasonic energy generated by the transducers. In other implementations the polymer substrate acts as an impedance matching layer improving the acoustic coupling between the ultrasound transducer and the surrounding tissue. The polymer backing 202 is a flexible, biocompatible material, such as silicone. In some implementations, the polymer backing 202 is manufactured with a shape memory such that the two tips 204 of the polymer backing 202 curl inward to roll around the target tissue. In some implementations, a shape memory alloy can be embedded in the cuff 102 to cause the cuff to wrap around the target tissue.

The cuff 102 also includes a plurality of electrodes 108. As illustrated the plurality of electrodes 108 are configured as an electrode array 208. The electrodes 108 of the electrode array 208 are configured as penetrating, depth electrodes. In some implementations, each of the depth electrodes contain a plurality of electrode sites along a length of the electrode shaft 210 of the electrodes 108. For example, the depth electrode can include a plurality of electrode sites along the electrode shaft 210 of each of the depth electrodes to provide readings (and stimulations) at different depths in the target tissue. As illustrated, the cuff 102 includes 16 electrode shafts 210. In other implementations, the cuff 102 includes between about 1 and about 256 electrode shafts 210, between about 4 and about 144 electrode shafts 210, between about 16 and about 100 electrode shafts 210, or between about 16 and about 64 electrode shafts 210. In some implementations, each electrode shaft 210 includes between about 1 and about 32 electrode sites, between about 1 and about 24 electrode sites, between about 1 and about 12 electrode sites, or between about 1 and about 6 electrode sites. The electrode shafts 210 project substantially perpendicular to a plane of the polymer backing 202.

The electrodes 108 of the cuff 102 can include platinum, platinum-iridium alloy, gold, iridium oxide, PEDOT, silver chloride, or a combination thereof. The transducers 106 can include bulk ferroelectric ceramics (e.g., diced slabs of polycrystalline lead zirconium titanate (PZT) or lead magnesium niobate-lead titanate (PMN-PT)), barium titanate, aluminum nitride, zinc oxide, tourmaline, berlinite, quartz, lithium tantalite, potassium niobate, bismuth iron oxide, crystal ferroelectrics, ferroelectric or electrostrictive polymers (e.g., polyvinylidene fluoride (PVDF)), capacitive micromachined ultrasonic transducers (cMUTs), piezoelectric micromachined ultrasonic transducers (pMUTs).

In some implementations, the transducers 106 can be used to both image and to stimulate the tissue 104. Stimulation of tissue, such as neurons, with ultrasonic energy from the transducers 106 can be advantageous in comparison to stimulating the tissue with only electrical energy from the electrodes 108. For example, ultrasonic energy may travel through connective and other fibrous tissue better than electrical energy. Because the patient's body naturally encapsulates foreign objects with a fibrous tissue electrical stimulation may become less effective over time; however, the ultrasonic energy can still be effective in stimulating target tissue even after the cuff 102 is encapsulated by the patient's body. Also, the effective stimulation zone of the ultrasonic energy can extend to greater depths when compared to the propagation waveform of electrical energy, enabling the ultrasonic waveform to stimulation more tissue than the electrical energy. The frequency of the ultrasonic energy can be selected to provide a predetermined penetration profile of the ultrasonic energy. For example, high frequency ultrasonic energy has a higher absorption rate and cannot penetrate as deeply when compared to low frequency ultrasonic energy, which has a lower absorption rate. In some implementations, over time the ultrasonic energy may cause less damage to the tissue when compared to electrical energy.

In some implementations, it is advantageous to include both transducers 106 and electrodes 108. For example, the electrodes 108 can be used to electrically stimulate the target tissue while the transducers 106 are used to image the target tissue. In another example where the target tissue is a nerve, the transducers 106 can be used to ultrasonically stimulate the nerve, and the electrodes 108 can be used to record the electrical potentials generated by the nerve in response to the ultrasonic stimulation.

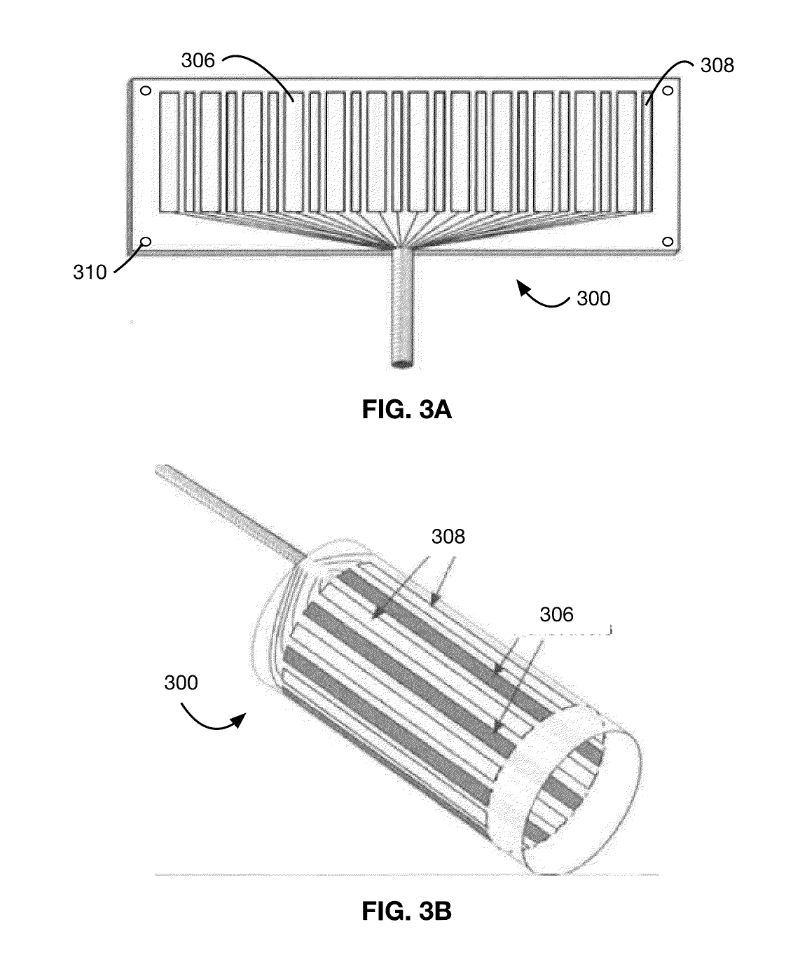

FIGS. 3A and 3B illustrate another example cuff 300 in a flat and rolled configuration, respectively. The cuff 300 includes a plurality of transducers 306 and a plurality of electrodes 308. The electrodes 308 and the transducers 306 alternate along the length of the cuff 300. The electrodes 308 of the cuff 300 are non-penetrating, flat electrodes. In some implementations, the electrodes 308 have a height between about 50 μm and about 5 mm, between about 250 μm and about 4 mm, between about 750 μm and about 3 mm, or between about 1 mm and about 2 mm. The electrodes 308 circular, square, rectangular, or other geometrically shaped face. The cuff 300 also includes a cutout 310 at each of the corners of the cuff 300. The cutouts 310 are used to secure the cuff 300 to the tissue. For example, sutures can be used to suture the cuff 300 to the tissue at each of the cutouts. Alternatively, once the cuff 300 is rolled around the target tissue, the rolled configuration (as illustrated in FIG. 3B) can be maintained by suturing the two top cutouts 308 together and the bottom two cutouts 308 together. By increasing or decreasing the slack of the suture between the pair of cutouts, the diameter of the rolled cuff can be configured to a desired diameter.

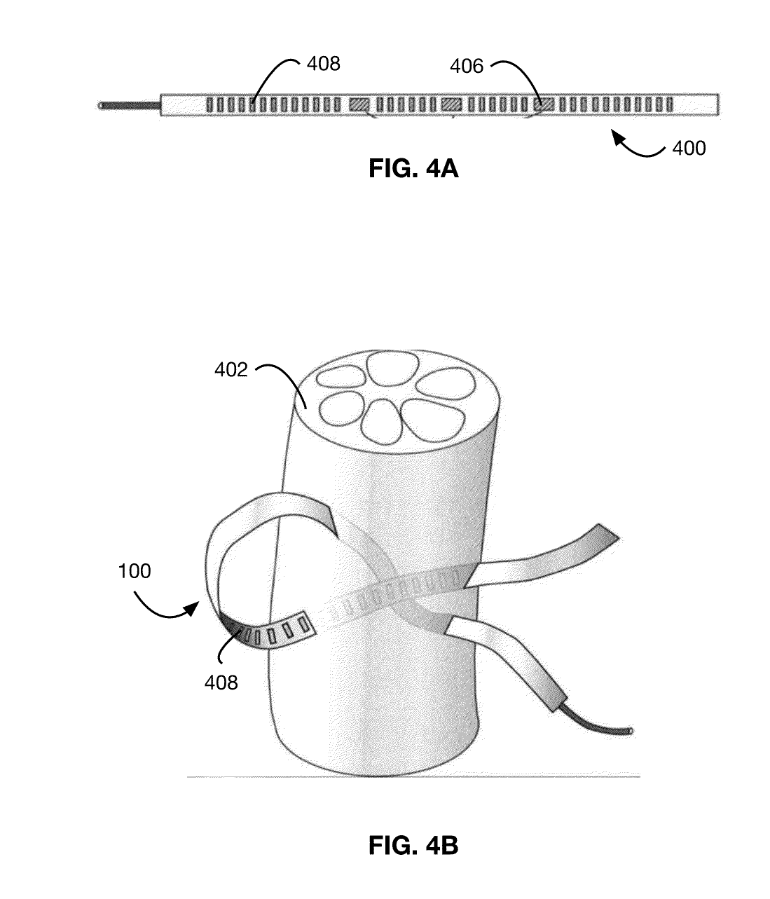

FIGS. 4A and 4B illustrate an example tissue interface 400 in a ribbon configuration with integral ultrasound transducers and electrodes. As described above in relation to the cuff configurations, the ribbon electrode 400 includes a plurality of transducers 406 and a plurality of electrodes 408. In some implementations, the ribbon electrode 400 is wrapped around the tissue. In other implementations, the ribbon electrode 400 is configured to pass through the target tissue 402. The ribbon electrode 400 can be passed through the target tissue 402 one or more times.

In some implementations, the tissue interfaces described herein are fabricated by coating a silicon wafer with a sacrificial layer, such as maltose. A first polymer layer is spin-coated onto the sacrificial layer and cured. A first metal layer that includes the electrodes and the transducers is then patterned onto the first polymer layer. The first metal layer can also include electrical traces and other circuitry, such as amplifiers and analog to digital converts. In some implementations, the tissue interface can include a plurality of metal layers separated by a dielectric material. For example, the tissue interface may include a first metal layer that acts as a routing layer and includes all of the traces and a second metal layer that includes the transducers and the electrodes. A second polymer layer can be spin-coated over the exposed first polymer layer and metal layer, encapsulating the metal layers. After the second polymer layer has cured, windows are made in the second polymer layer with laser ablation or other machining process to expose the faces of the transducers and electrodes. The completed tissue interface is removed from the silicon wafer by dissolving the sacrificial layer.

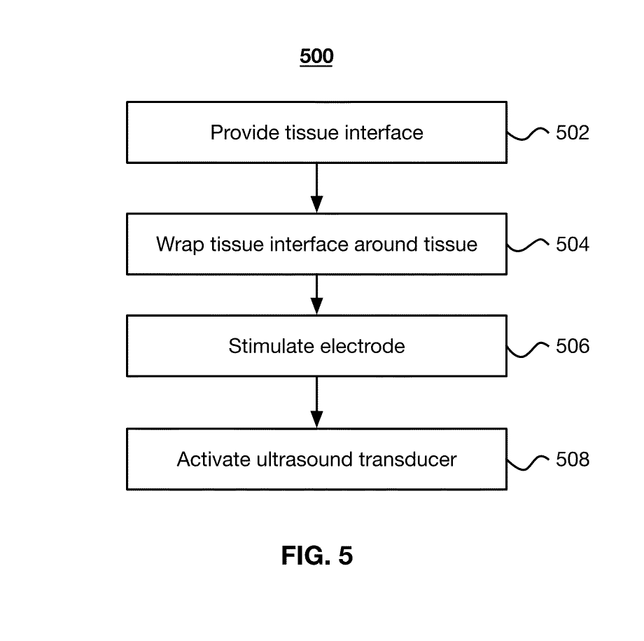

FIG. 5 illustrates a flow chart of an example method 500 for stimulating a target tissue. The example method 500 includes providing a tissue interface (step 502). The tissue interface is wrapped around the target tissue (step 504). The electrodes of the tissue interface are stimulated (step 506) and the ultrasound transducers of the tissue interface are activated (step 508).

As set forth above, the method 500 includes providing a tissue interface (step 502). The tissue interface can be any of the tissue interfaces described herein. For example, the tissue interface can be configured as a cuff electrode that is configured to wrap around a nerve or other neural tissue. In some implementations, the tissue interface is in a ribbon configuration and is designed to be inserted into the target tissue. The target tissue can, for example, be a muscle, central nervous tissue, or peripheral nervous tissue. The tissue interface includes a plurality of electrode sites and ultrasound transducers. In some implementations, the electrodes are disk or other configurations of a planar electrode. In other implementations, the electrodes are depth electrodes that include one or more electrode sites along the shaft of each of the depth electrodes.

The method 500 also includes wrapping the tissue interface around the target tissue (step 504). In some implementations, the tissue interface is physically secured to the target tissue by sutures, surgical glue, or clamps. For example, if a patient has a spinal injury that prevents the patient from activating a muscle, the tissue interface may be implanted around the nerve that innervates the muscle distal to the damaged neural tissue. The tissue interface can be used to stimulate the innervating nerve—bypassing the damaged portion of the nerve.

After implantation of the tissue interface the electrodes are stimulated (step 506) and the ultrasound transducers are activated (step 508). The electrodes of the tissue interface are activated by the ESR, which sends electrical pulses or waves to the electrodes to stimulate the target tissue. In some implementations, the ESR configures which of plurality of electrodes act as stimulating electrodes and which of the plurality of electrodes act as recording electrodes. Similarly, the TSR activates the ultrasound transducers to deliver ultrasonic energy to the target tissue. The ultrasound transducers are activated when the TSR sends a series of electrical pulses to the ultrasound transducers, which cause the ultrasound transducers to resonant. The frequency of the ultrasonic energy is controlled by the TSR and the frequency is selected to control the amount of penetration that the ultrasonic energy has into the target tissue. In some implementations, the ultrasonic energy is configured to elicit a response from the target tissue (e.g., activate a neuron) and in other implementations the ultrasonic energy is configured to provide an A-mode or B-mode ultrasound image of the target tissue.

The disclosed system and methods may be embodied in other specific forms without departing from the spirit or essential characteristics thereof. The forgoing implementations are therefore to be considered in all respects illustrative, rather than limiting of the invention.

What is claimed:

1. A tissue interface device comprising:
a biocompatible polymer backing;
an electrode array coupled to the biocompatible polymer backing, the electrode array comprising one or more electrode shafts projecting substantially perpendicular to a plane of the biocompatible polymer backing, wherein each of the one or more electrode shafts comprise an electrode site; and
one or more ultrasound transducers coupled to the biocompatible polymer backing.

2. The tissue interface device of claim 1, wherein each of the one or more electrode shafts further comprise a plurality of electrode sites distributed along a length of the respective one or more electrode shafts.

3. The tissue interface device of claim 1, wherein the one or more ultrasound transducers each comprise at least one of a bulk ferroelectric ceramic, a crystal ferroelectric, a ferroelectric polymer, a capacitive micromachined ultrasonic transducer (cMUT), a piezoelectric micromachined transducer (pMUT), and an electrostrictive polymer.

4. The tissue interface device of claim 1, further comprising a controller configured to energize the one or more ultrasound transducers at a first frequency for stimulating a target and at a second frequency for imaging the target.

5. The tissue interface device of claim 4, wherein the controller is further configured to record an electrical signal detected at the electrode site.

6. The tissue interface device of claim 1, further comprising a rechargeable battery.

7. The tissue interface device of claim 6, further comprising an induction coil to wirelessly receive energy from an external source and provide the received energy to the rechargeable battery.

8. The tissue interface device of claim 1, wherein the electrode array comprises between about 2 and about 64 electrode shafts.

9. The tissue interface device of claim 1, wherein the biocompatible polymer backing is a flexible cuff configured to wrap around a nerve.

10. A method comprising:
coupling a tissue interface device to a target tissue, the tissue interface device comprising:
a biocompatible polymer backing;
one or more electrodes coupled to the biocompatible polymer backing; and
one or more ultrasound transducers coupled to the biocompatible polymer backing;
delivering a stimulation signal to the target tissue via the one or more electrodes;
imaging the target tissue via the one or more ultrasound transducers.

11. The method of claim 10, further comprising ultrasonically stimulating the target tissue via the one or more ultrasound transducers.

12. The method of claim 11, further comprising imaging the target tissue via the one or more ultrasound transducers with a first frequency of ultrasonic energy and ultrasonically stimulating the target tissue via the one or more ultrasound transducers with a second frequency of ultrasonic energy different than the first frequency of ultrasonic energy.

13. The method of claim 10, wherein imaging the target tissue further comprises detecting a scar formation on the target tissue.

14. The method of claim 10, wherein the tissue interface device is configured as a cuff and coupling the tissue interface device to the target tissue further comprises wrapping the tissue interface device around the target tissue.

15. The method of claim 10, wherein the one or more electrodes each comprise an electrode shaft projecting substantially perpendicular to a plane of the biocompatible polymer backing, wherein each electrode shaft comprises an electrode site.

16. The method of claim 15, wherein each electrode shaft further comprise a plurality of electrode sites distributed along a length of the electrode shaft.

17. The method of claim 15, wherein the tissue interface device comprises between about 2 and about 64 electrode shafts.

18. The method of claim 10, wherein the target tissue is neural tissue.

19. The method of claim 10, wherein the one or more ultrasound transducers comprise at least one of a bulk ferroelectric ceramic, a crystal ferroelectric, a ferroelectric polymer, a capacitive micromachined ultrasonic transducer (cMUT), a piezoelectric micromachined transducer (pMUT), and an electrostrictive polymer.

20. The method of claim 10, further comprising wirelessly charging a battery of the tissue interface device.
